(19) United States
(12) Patent Application Publication
(10) Pub. No.: US 2010/0161069 A1
(43) Pub. Date: Jun. 24, 2010

(54) ASSEMBLY CONSISTING OF HEAD AND MOVABLE NECK FOR PREPARING THE IMPLANTATION OF AN ARTIFICIAL HIP

(75) Inventor: Sheila Ragbir, Whim (TT)

Correspondence Address: YOUNG & THOMPSON, 209 Madison Street, Suite 500, Alexandria, VA, 22314, US

(73) Assignee: MARIASAL INVESTMENT N.V.

(21) Appl. No.: 12/296,135

(22) PCT Filed: Apr. 5, 2006
(86) PCT No.: PCT/IB06/00791

§ 371 Date: Oct. 6, 2008

(51) Int. Cl. A61F 2/32

(52) U.S. Cl. 623/22.11

(57) ABSTRACT

An assembly including a head and movable neck for preparing the implantation of an artificial hip is described, the head and the movable neck having, respectively, a frustoconical hole and a frustoconical portion insertable therein. The flat end of the frustoconical portion has, coaxially formed or mounted thereon, a cylindrical protrusion having a diameter smaller than that of the frustoconical portion opposite its fiat end and the frustoconical hole has a coaxial, blind, cylindrical extension from the side walls of which an annular protrusion having an internal diameter which produces a slight interference with the diameter of the cylindrical protrusion projects, and the height of the cylindrical extension is greater than or equal to that of the cylindrical protrusion so that, when the latter is inserted over its entire height inside the cylindrical extension, the flat end bears against a concentric shoulder formed on the head around the cylindrical extension.

The present invention relates to the technical sector which deals with the production of the various components necessary for performing the implantation of an artificial hip.

More particularly, it relates to those components of an artificial hip, i.e. a movable neck on which a spherical head is mounted, which are made of plastic material and are used for tests and measurements during an operation in order to define as precisely as possible the dimensions and the positioning of the abovementioned parts of the final artificial hip which must be implanted.

These components made of plastic material are faithful reproductions of a real movable neck and real spherical head and, as such, are assembled together by means of a conical fit between a frustoconical hole formed in the head and a frustoconical portion of the neck which is inserted in a complementary manner inside it.

This configuration of the parts is such that, very frequently, when handling them in order to simulate an operation, forced engagement occurs between their frustoconical surfaces which makes disengagement thereof awkward and difficult, with complications which are easy to imagine and a loss of time which is prejudicial in a surgical environment.

In order to overcome this drawback, the inventor of the subject of the present application has devised a “facsimile” of an artificial hip which, when used, avoids the risk of forced engagement mentioned above owing to various constructional measures which will be described more fully below.

In the spherical head forming part of the assembly of components which simulate an artificial hip according to the invention, the said frustoconical hole has a cylindrical extension from the side walls of which an annular protrusion projects slightly, and the flat end of the frustoconical portion of the neck has, projecting from it, a coaxial cylindrical protrusion with a slightly smaller diameter which may be introduced coaxially into the abovementioned extension of the frustoconical hole in the head, remaining constrained there as a result of the interference caused by the existence of the said annular protrusion and owing to the elasticity of the plastic materials with which the abovementioned so-called “test” neck and head are made.

The height of the said cylindrical protrusion in the axial direction is less than or the same as that of the said extension so that, when it is inserted fully inside the latter, the flat surface of the end of the frustoconical portion of the neck bears against a concentric shoulder formed in the head around the abovementioned cylindrical extension of its frustoconical hole before engagement occurs between the inclined surfaces of the frustoconical portion of the neck and the frustoconical hole in the head.

An undesirable forced engagement between the various components is thus avoided in a sure and reliable manner and the possibility is provided of being able to replace easily the said components during the preliminary operations which precede the fitting of the final prostheses.

The present invention therefore relates to an assembly consisting of head and movable neck for simulating the modular parts of an artificial hip as described in the accompanying claim 1.

A preferred example of embodiment of the assembly according to the invention will now be described in more detail with reference also to the accompanying drawings in which:

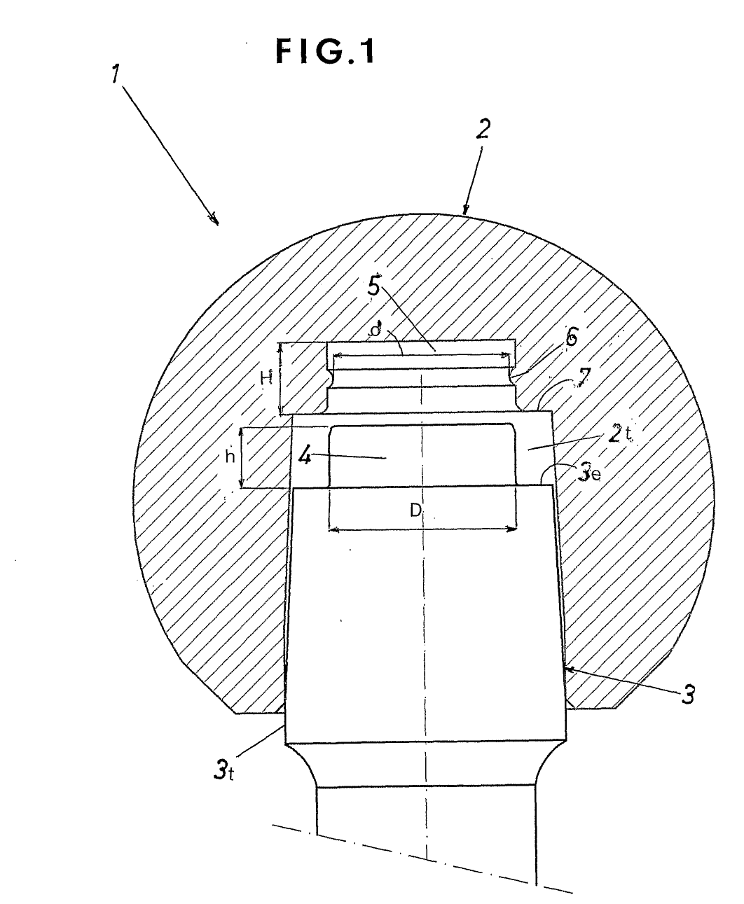

FIG. 1 is a longitudinal section through the said example of embodiment of an assembly according to the invention, with a movable neck introduced only partly into the frustoconical hole of a spherical head;

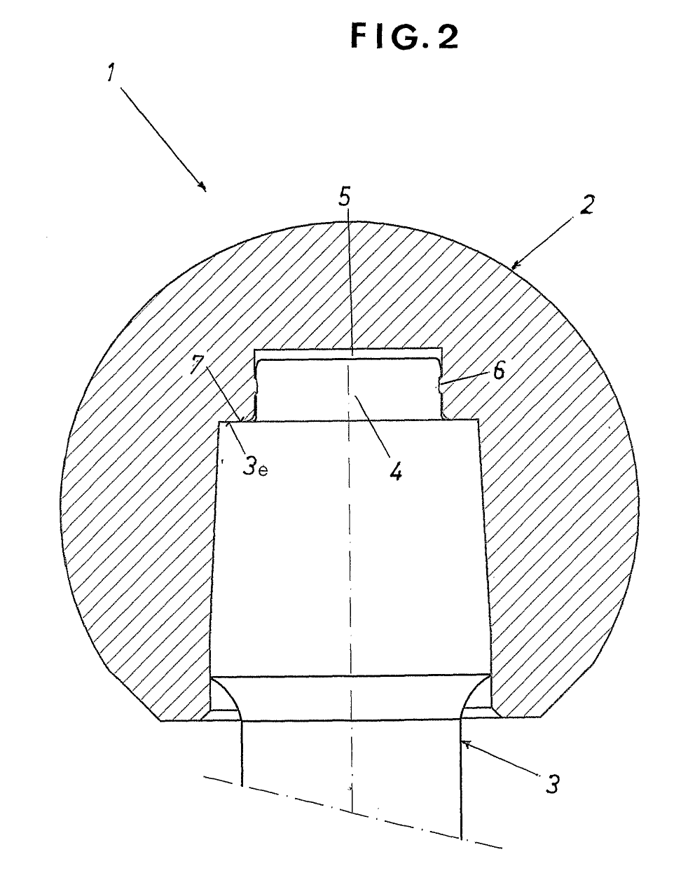

FIG. 2 is the same longitudinal section as in FIG. 1, with the movable neck introduced completely into the said frustoconical hole of the head inside which it remains constrained.

If we consider FIG. 1 firstly, in this figure it can be seen how, in an assembly 1 according to the invention, a spherical head 2 has a frustoconical hole 2t provided with a blind, coaxial, cylindrical extension 5, from the side walls of which an annular protrusion 6 (enlarged in the drawing for the sake of illustrative clarity) projects inwards. A flat shoulder 7, which is concentric therewith, is formed around the base of this cylindrical extension 5.

The flat end 3e of the frustoconical portion 3t of a movable neck 3 has, coaxially formed thereon, a cylindrical protrusion 4 having a diameter D smaller than that of the said frustoconical portion 3t opposite its flat end 3e.

As can be seen in the drawing, the internal diameter d of the abovementioned annular protrusion 6 is slightly smaller than, or rather substantially equal to, with slight interference, the diameter D of the abovementioned cylindrical protrusion 4 of the frustoconical portion 3t of the neck 3, and, the height H of the said cylindrical extension 5 is also greater than or equal to the height h of the cylindrical protrusion 4 and is calculated so that, when the latter is inserted over its entire height inside the said cylindrical extension 5, as shown in FIG. 2, the said flat end 3e of the frustoconical portion 3t of the neck 3 bears against the already mentioned shoulder 7 formed on the head 2 concentrically with respect to the said cylindrical extension 5 of its frustoconical hole 2t, preventing forced engagement occurring between the inclined surfaces of the parts.

By means of the already mentioned slight interference between the said annular cylindrical protrusion 6 and the said cylindrical protrusion 4 of the neck 3 the parts may be fixed together in a sufficiently reliable manner to perform the already described preliminary operations, with the possibility of easily disengaging them with minimum effort

The predefined object of the inventor has thus been achieved.

1. Assembly (1) consisting of head (2) and movable neck (3) for preparing the implantation of an artificial hip, the said head (2) and the said movable neck (3) having, respectively, a frustoconical hole (2t) and a frustoconical portion (3t) able to be inserted in a complementary manner therein, characterized in that the flat end (3e) of the said frustoconical portion (3t) has, coaxially formed or mounted thereon, a o cylindrical protrusion (4) having a diameter (D) smaller than the diameter of the frustoconical portion (3t) opposite its said flat end (3e) and in that the said frustoconical hole (2t) has a coaxial, blind, cylindrical extension (5) from the side walls of which an annular protrusion (6) having an internal diameter (d) which produces a slight interference with the diameter (D) of the said cylindrical protrusion (4) of the frustoconical portion (3t) projects, and the height (H) of the said cylindrical extension (5) of the frustoconical hole (2t) is greater than or equal to the height (h) of the abovementioned cylindrical protrusion (4) so that, when the latter (4) is inserted over its entire height inside the said cylindrical extension (5), the said flat end (3e) of the frustoconical portion (3t) bears against a concentric shoulder (7) formed on the head (2) around the said cylindrical extension (5) before forced engagement occurs between the surfaces of the frustoconical portion (3t) of the neck (3) and the frustoconical hole (2t) in the head (2).

2. Assembly according to claim 1, in which the profile of the section of the said annular protrusion (6) has the form of a circle arc.
